(19) United States
(12) Patent Application Publication
(10) Pub. No.: US 2023/0023521 A1
(43) Pub. Date: Jan. 26, 2023

(54) INTERVENTIONAL PROCEDURE OPTIMIZATION

(71) Applicant: KONINKLIJKE PHILIPS N.V., EINDHOVEN (NL)

(72) Inventors: Torre Michelle BYDLON, Melrose, MA (US); Amir Mohammad TAHMASEBI MARAGHOOSH, Arlington, MA (US); Shawn Arie Peter STAPLETON, Seattle, WA (US)

(21) Appl. No.: 17/770,652

(22) PCT Filed: Oct. 13, 2020
(86) PCT No.: PCT/EP2020/078693

§ 371 Date: Apr. 21, 2022

Related U.S. Application Data
(60) Provisional application No. 62/923,645, filed on Oct. 21, 2019.

(51) Int. Cl. G16H 20/40; G16H 15/00; G16H 50/30

(52) U.S. Cl. CPC G16H 20/40; G16H 15/00; G16H 50/30

(57) ABSTRACT

A controller (122, 910/920) for interventional procedure optimization includes a processor (12210, 910) and a memory (12220, 920) that stores instructions. When executed by the processor, the instructions cause the controller (12210, 910) to implement a process that includes identifying (S210) anatomical characteristics from pre-interventional imagery of anatomy for each of multiple candidate types of an interventional procedure for the anatomy and comparing (S220) the anatomical characteristics with tool characteristics of candidate tools to use in each of the candidate types. The process also includes generating (S240) a feasibility report for each of the candidate types based on the identifying and the comparing. Each feasibility report includes a feasibility grade for each of the candidate types. The process also includes selecting (S260), based on the feasibility reports, an optimal interventional procedure type among the candidate types. An interventional procedure is performed on the anatomy using the optimal interventional procedure type based on the selecting (S260).

### BACKGROUND

Lung cancer is the deadliest form of cancer worldwide today. Several countries have implemented lung cancer screening programs to detect lung cancer at earlier stages. Several treatment options are available for early stage lung cancer and result in improved 5-year survival rates. For patients suspected of having lung cancer, whether through screening or other means, it is essential to obtain a diagnosis of suspicious lung tissue. Lung tissue can be obtained through several types of interventional procedures including by an endobronchial biopsy, a transthoracic biopsy or a surgical biopsy. Endobronchial biopsy is the preferred type of biopsy to obtain a sample of lung tissue for diagnosis because complication rates are low. However, the diagnostic yield for an endobronchial biopsy can be as low as 30% for peripherally located lung cancer nodules. Transthoracic biopsy and surgical biopsy have much higher diagnostic yields but complication rates are higher.

Sixty percent or more of lung cancer patients may require at least one biopsy to obtain a lung tissue sample of diagnostic quality. Typically an endobronchial biopsy is first attempted, in order to minimize complications. If the endobronchial biopsy fails to yield a suitable lung tissue sample, then patients may be sent for a transthoracic biopsy or a surgical biopsy, both of which carry a higher risk of complications. Hence, complication risks are balanced with the possibility of obtaining a good lung tissue sample with a higher diagnostic yield.

Artificial intelligence (AI) is used in lung cancer screening such as with imaging analysis to automatically detect and locate suspected lung cancer nodules in computed tomography (CT) imagery. Information about the size, appearance, and growth rates is used to establish a threshold for whether suspicious tissue should be biopsied as part of the lung cancer screening. However, there are currently no established guidelines or support tools to provide decision support regarding the type of biopsy a patient should undergo. Interventional procedure optimization described herein addresses these challenges.

### SUMMARY

According to an aspect of the present disclosure, a controller for interventional procedure optimization includes a memory and a processor. The memory stores instructions. The processor executes the instructions. When executed by the processor, the instructions cause the controller to implement a process that includes identifying anatomical characteristics from pre-interventional imagery of anatomy for each of a plurality of candidate types of interventional procedures for the anatomy and comparing the anatomical characteristics with tool characteristics of candidate tools to use in each of the plurality of candidate types. The process implemented by the controller when the instructions are executed by the processor also includes generating, based on the identifying and the comparing, a feasibility report for each of the plurality of candidate types. Each feasibility report includes a feasibility grade for each of the plurality of candidate types. The process implemented by the controller when the instructions are executed by the processor also includes selecting, based on the feasibility report for each of the plurality of candidate types, an optimal interventional procedure type among the plurality of candidate types. An interventional procedure is performed on the anatomy using the optimal interventional procedure type based on the selecting.

According to another aspect of the present disclosure, an apparatus for interventional procedure optimization includes an input interface and a controller. The input interface inputs pre-interventional imagery of anatomy. The controller includes a memory and a processor. The memory stores instructions. The processor executes the instructions. When executed by the processor, the instructions cause the controller to implement a process. The process implemented by the controller when the processor executes the instructions includes identifying anatomical characteristics from the pre-interventional imagery of anatomy for each of a plurality of candidate types of interventional procedures for the anatomy and comparing the anatomical characteristics with tool characteristics of candidate tools to use in each of the plurality of candidate types. The process implemented by the controller when the processer executes the instructions also includes generating, based on the identifying and the comparing, a feasibility report for each of the plurality of candidate types. Each feasibility report includes a feasibility grade for each of the plurality of candidate types. The process implemented by the controller when the processer executes the instructions further includes selecting, based on the feasibility report for each of the plurality of candidate types, an optimal interventional procedure type among the plurality of candidate types. An interventional procedure is performed on the anatomy using the optimal interventional procedure type based on the selecting.

According to yet another aspect of the present disclosure, a system for interventional procedure optimization includes an input interface, a monitor and a controller. The input interface inputs pre-interventional imagery of anatomy. The monitor displays the pre-interventional imagery of anatomy. The controller includes a memory and a processor. The memory stores instructions. The processor executes the instructions. When executed by the processor, the instructions cause the controller to implement a process that includes identifying anatomical characteristics from the pre-interventional imagery of anatomy for each of a plurality of candidate types of an interventional procedure for the anatomy and comparing the anatomical characteristics with tool characteristics of candidate tools to use in each of the plurality of candidate types. The process implemented by the controller when the processor executes the instructions also includes generating, based on the identifying and the comparing, a feasibility report for each of the plurality of candidate types. Each feasibility report includes a feasibility grade for each of the plurality of candidate types. The process implemented by the controller when the processer executes the instructions also includes selecting, based on the feasibility report for each of the plurality of candidate types, an optimal interventional procedure type among the plurality of candidate types. An interventional procedure is performed on the anatomy using the optimal interventional procedure type based on the selecting.

### BRIEF DESCRIPTION OF THE DRAWINGS

The example embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1A illustrates a system for interventional procedure optimization, in accordance with a representative embodiment.

FIG. 1B illustrates a controller for interventional procedure optimization, in accordance with a representative embodiment.

FIG. 2 illustrates a method for interventional procedure optimization, in accordance with a representative embodiment.

FIG. 3 illustrates an artificial intelligence overview for interventional procedure optimization, in accordance with a representative embodiment.

FIG. 4 illustrates a controller for generating a feasibility report for interventional procedure optimization, in accordance with a representative embodiment.

FIG. 5 illustrates path planning for an interventional procedure in interventional procedure optimization, in accordance with a representative embodiment.

FIG. 6 illustrates a feasibility map for interventional procedure optimization, in accordance with a representative embodiment.

FIG. 7 illustrates path planning for another interventional procedure in interventional procedure optimization, in accordance with a representative embodiment.

FIG. 8 illustrates another feasibility map for interventional procedure optimization, in accordance with a representative embodiment.

FIG. 9 illustrates a computer system, on which a method for interventional procedure optimization is implemented, in accordance with another representative embodiment.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms ‘a’, ‘an’ and ‘the’ are intended to include both singular and plural forms, unless the context clearly dictates otherwise. Additionally, the terms “comprises”, and/or “comprising,” and/or similar terms when used in this specification, specify the presence of stated features, elements, and/or components, but do not preclude the presence or addition of one or more other features, elements, components, and/or groups thereof. As used herein, the term “and/or” includes any and all combinations of one or more of the associated listed items.

Unless otherwise noted, when an element or component is said to be “connected to”, “coupled to”, or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

The present disclosure, through one or more of its various aspects, embodiments and/or specific features or sub-components, is thus intended to bring out one or more of the advantages as specifically noted below. For purposes of explanation and not limitation, example embodiments disclosing specific details are set forth in order to provide a thorough understanding of an embodiment according to the present teachings. However, other embodiments consistent with the present disclosure that depart from specific details disclosed herein remain within the scope of the appended claims. Moreover, descriptions of well-known apparatuses and methods may be omitted so as to not obscure the description of the example embodiments. Such methods and apparatuses are within the scope of the present disclosure.

As described herein, automated determinations of optimal types of interventional procedures such as types of biopsies and ablation are provided in a process based on a feasibility analysis of each of multiple different types of interventional procedures. The different types of interventional procedures under consideration are also referred to as candidate types. The analysis involves tradeoffs such as maximizing diagnostic yield while minimizing complications. A management tool may leverage artificial intelligence (AI) to integrate multi-modal data, including, but not limited to, imaging, medical risks, procedure cost, operator experience, and technical feasibility, to quantify for each candidate type the success rate and a risk factor score. The automated determinations can be used to improve patient management such that an interventional procedure is performed right the first time. In the case of different types of biopsies as the candidate types of interventional procedures, the automated determination may result in tissue sampling that is of diagnostic quality, while minimizing risks of complications, and in a cost effective manner.

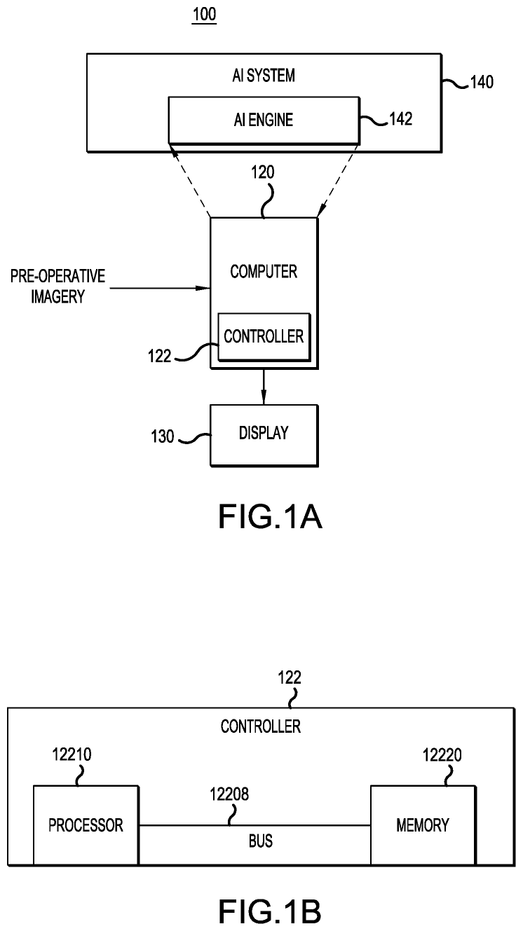

FIG. 1A illustrates a system for interventional procedure optimization, in accordance with a representative embodiment.

The system 100 in FIG. 1A is a system for interventional procedure optimization and includes components that may be provided together or that may be distributed. The system 100 includes a computer 120, a display 130 and an AI system 140. The computer 120 includes a controller 122. The AI system 140 includes an AI engine 142. The computer 120 may be local to the display 130 and may be connected to the display 130 via a local wired interface such as an Ethernet cable or via a local wireless interface such as a Wi-Fi connection. The computer 120 may be remote from the AI system 140 and may be connected to the AI system 140 via the internet using one or more wired connection(s) and/or wireless connection(s).

The computer 120 may include one or more input interface(s). The input interfaces (not shown) of the computer 120 may include ports, disk drives, wireless antennas, or other types of receiver circuitry. The input interfaces may further connect user interfaces, such as a mouse, a keyboard, a microphone, a video camera, a touchscreen display, or another element or component to the computer 120. The input interfaces of the computer 120 may receive input of pre-interventional imagery of anatomy before the interventional procedure as well as interventional imagery during the interventional procedure. One or more input interface(s) may also receive input to select or deselect a candidate type of interventional procedure; a candidate tool to use in the candidate type of interventional procedure; and a path through the anatomy to reach a target site in the candidate type of the interventional procedure. For example, an input interface of the computer 120 may connect a mouse that controls a cursor used to identify and select a candidate type, a candidate tool, and/or one of multiple possible different paths to reach a target of the interventional procedure. Alternatives to a mouse connected to the computer 120 include voice or gesture recognition captured by a microphone or video camera connected to the computer 120.

The display 130 may be a monitor such as a computer monitor, a display on a mobile device, a television, an electronic whiteboard, or another screen configured to display electronic imagery. The display 130 may also include one or more input interface(s) such as those noted above that may connect other elements or components to the computer 120, as well as a touch screen that enables direct input via touch. For example, the selections and/or deselections of candidate type of the interventional procedure, candidate tool(s) to use in the candidate type of the interventional procedure, and path through the anatomy, may all be made via a touch screen input interface of the display 130. The display 130 may display the pre-interventional imagery of anatomy and the interventional imagery. For example, the computer 120 may retrieve or otherwise receive pre-interventional imagery such as computed tomography imagery, MR imagery, and/or ultrasound imagery, via an input interface from a website, an email, a portable disk or other type of memory. The computer 120 may provide the pre-interventional imagery such as computed tomography imagery to the display 130 via the local wired interface or local wireless interface as pre-interventional imagery for display on the display 130. The computer 120 may also receive the interventional imagery such as x-ray or ultrasound imagery, such as over a wired connection from imaging machines and systems that operate to generate the interventional imagery, and provide the interventional imagery to the display 130 for displaying.

The controller 122 of the computer 120 in the system 100 may include a memory (see FIG. 1B) that stores instructions and a processor (see FIG. 1B) that executes the instructions. When executed by the processor, the instructions cause the controller 122 to implement a process that includes identifying anatomical characteristics from the pre-interventional imagery of anatomy for each of multiple candidate types of an interventional procedure for the anatomy. The process implemented by the controller 122 based on the processor executing the instructions also includes comparing the anatomical characteristics with tool characteristics of candidate tools to use in each of the multiple candidate types. The process implemented by the controller 122 also includes generating, based on the identifying and the comparing, a feasibility report for each of the multiple candidate types. Each feasibility report may include a feasibility grade that may be weighted differently for each of the multiple candidate types. The process implemented by the controller 122 also may include selecting, based on the feasibility report for each of the multiple candidate types, an optimal interventional procedure type among the multiple candidate types. The feasibility report generated by the controller 122 may also define which interventional tool(s) are best predicted to successfully reach a target site in the optimal interventional procedure type. An interventional procedure is performed on the anatomy using the optimal interventional procedure type based on the selecting. The interventional procedure may be performed based on the selected optimal interventional tool(s).

The implementation of the process by the controller 122 may include one or more of the above-noted operations implemented based on the processor executing instructions. For example, the controller 122 may directly perform the identifying of anatomical characteristics, the comparison of anatomical characteristics with tool characteristics, the generating of the feasibility report, and the selecting of an optimal interventional procedure type. The implementation of the process by the controller 122 may also include other operations that are indirectly implemented by the controller 122, such as by instructing or otherwise communicating with another element of the system 100, such as the display 130 or the AI system 140, to perform one or more of the above-noted operations or other operations. For example, the controller 122 may provide imagery and instructions to the display 130 so that the display 130 displays selected information such as choices of interventional procedures and tools to use in each interventional procedure. Similarly, insofar as the controller 122 is an element of the computer 120 as an apparatus and the computer 120 is an element of the system 100, the operations attributed to the controller 122 above may also be attributed to the computer 120 as an apparatus for interventional procedure optimization and to the system 100 as a system for interventional procedure optimization.

The AI system 140 performs machine learning based on pre-interventional imagery, feasibility reports corresponding to the pre-interventional imagery, and clinical outcomes of interventional procedures performed based on the feasibility reports. The AI system 140 may also receive data from the interventional procedure, such as sensor data from sensor-equipped tools used in the interventional procedure. As mentioned above, the AI system 140 includes the AI engine 142, and generates and implements artificial intelligence based on the machine learning. The AI engine 142 is implemented as software that implements and applies the machine learning described herein. The AI system 140 may implement the machine learning and the artificial intelligence in a cloud, such as at a data center, for example, in which case the AI system 140 may be connected to the computer 120 via the internet using one or more wired and/or wireless connection(s). The AI system 140 may be connected to multiple different computers including the computer 120, so that the machine learning and artificial intelligence are performed centrally based on and for a relatively large set of interventional procedures for different patients at different locations. Alternatively, the AI system 140 may implement the machine learning and the artificial intelligence locally to the computer 120, such as at a facility that performs interventional procedures of a similar nature (e.g., lung cancer interventional procedures) for large numbers of patients.

FIG. 1B illustrates controller 122 for interventional procedure optimization, in accordance with a representative embodiment.

The controller 122 in FIG. 1B is an interventional procedure optimization controller and may be provided as a stand-alone component as shown or as a component of a device such as the computer 120 in FIG. 1A. The controller 122 includes a processor 12210, a memory 12220 and a bus 12208. The processor 12210 retrieves or otherwise receives instructions from the memory 12220 via the bus 12208.

As described herein, the controller 122 is provided for interventional procedure optimization. The memory 12220 stores instructions and the processor 12210 executes the instructions. When executed by the processor 12210, the instructions cause the controller 122 to implement a process that includes identifying anatomical characteristics from pre-interventional imagery of anatomy for each of multiple candidate types of an interventional procedure for the anatomy. The process implemented by the controller 122 when the processor 12210 executes the instructions also includes comparing the anatomical characteristics with tool characteristics of candidate tools to use in each of the multiple candidate types. The process implemented by the controller 122 also includes generating, based on the identifying and the comparing, a feasibility report for each of the multiple candidate types of interventional procedure. The process implemented by the controller 122 may also include identifying tool(s) which is/are considered for use in each of the multiple candidate types of interventional procedure, and the identified tool(s) may be included in the feasibility report. Each feasibility report includes a feasibility grade that may be weighted differently for each of the multiple candidate types. The weightings for the feasibility grade vary based on an expected diagnostic yield that varies for each of the different candidate types. The weightings for the feasibility grade may be set based on applying artificial intelligence to previous instantiations of similar interventional procedures, such as based on the diagnostic yields of the previous instantiations.

The process implemented by the controller 122 also includes selecting, based on the feasibility report for each of the multiple candidate types, an optimal interventional procedure type among the multiple candidate types. The process implemented by the controller 122 may also include selecting one or more optimal interventional tools to use to reach the target site in the optimal interventional procedure type. An interventional procedure is performed on the anatomy using the selected optimal interventional procedure type. The interventional procedure may be performed using the selected optimal interventional tool(s).

The controller 122 may perform some of the operations described herein directly and may implement other operations described herein indirectly. For example, the controller 122 may directly identify anatomical characteristics from pre-interventional imagery, may directly compare the anatomical characteristics with tool characteristics, may directly generate a feasibility report for each of multiple candidate types, and/or may directly select an optimal interventional procedure type, as well as optimal tool(s) to use. The controller 122 may indirectly control other operations such as by initiating a request for the AI system 140 to apply artificial intelligence and initiating the procedures that will result in the interventional procedure being performed. Accordingly, the process implemented by the controller 122 when the processor 12210 executes instructions from the memory 12220 may include steps not directly performed by the controller 122.

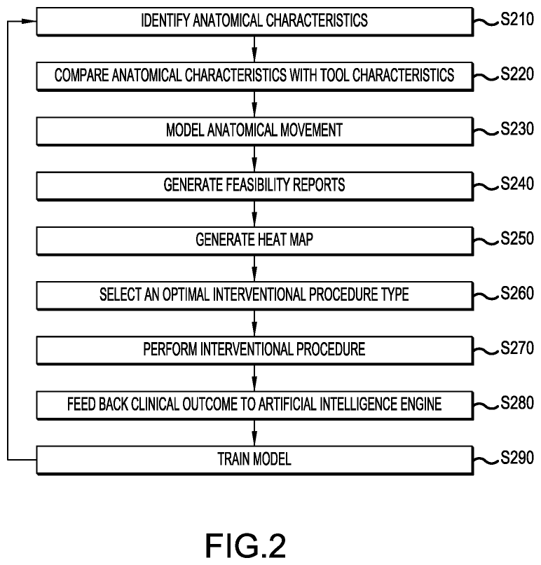

FIG. 2 illustrates an interventional procedure optimization process, in accordance with a representative embodiment. The method of FIG. 2 may be performed by a single apparatus, a single system, by or on behalf of a single entity, or by distributed apparatuses, distributed systems, or by or on behalf of multiple entities.

At S210 of FIG. 2, anatomical characteristics are identified. The anatomical characteristics may be identified by the controller 122 of the computer 120 in FIG. 1A, such as based on the pre-operative imagery that is received by the computer 120 in FIG. 1A. The anatomical characteristics may include organs, bones, vessels, airways, lung cancer tumors/nodules, and other parts of human anatomy. The anatomical characteristics may also include mechanical characteristics of anatomy, such as diameters, curvatures, elasticity of airways, elasticity of tissues surrounding the anatomy subject to the interventional procedure, and so on. For example, the anatomical characteristics may include a diameter of an airway, a curvature of an airway, an elasticity of tissue such as an airway or an elasticity of surrounding tissues surrounding the anatomy to be subjected to the interventional procedure. The anatomical characteristics may also include a relative location of a target of the interventional procedure in the anatomy and one or more paths to the relative location of the target. The relative location may be relative to an origin of a three-dimensional coordinate system used in the interventional procedure. The mechanical characteristics of tissue may be specifically compared to mechanical characteristics and capabilities of different potential tools to be used in an interventional procedure, as explained below with respect to S220.

The anatomical characteristics may be identified from pre-interventional imagery of anatomy such as computed tomography imagery. The anatomical characteristics may be identified for each of multiple candidate types of an interventional procedure for the anatomy. As an example, the interventional procedure may be a biopsy and the multiple candidate types may include an endobronchial biopsy of lung tissue, a transthoracic biopsy of lung tissue through a thoracic cavity, and a surgical biopsy of lung tissue performed by surgery. Accordingly, S210 may involve identifying anatomical characteristics from pre-interventional imagery of anatomy for each of multiple candidate biopsy types or other types of an interventional procedure for the anatomy. The anatomical characteristics may vary for each different type of interventional procedure, such as when paths to a target location will vary and encounter different anatomical tissue in two different types of interventional procedure.

In an embodiment, the identification of anatomical characteristics at S210 may include or may be followed by automatically detecting a target location for the interventional procedure. The target location may be a tumor identified as an anatomical characteristic from image analysis at S210. The image analysis may first identify recognizable anatomical characteristics, and then automatically identify one or more of the identified anatomical characteristics as a target location. The identified anatomical characteristics may also be labelled or marked by annotations in the pre-interventional imagery, such as by a predesignated set of symbols to label a lung, a heart, and suspected tumors in the tissue of the lung and/or the heart.

At S220, the anatomical characteristics are compared with tool characteristics of candidate tools. The anatomical characteristics may be compared with tool characteristics at S220 by the controller 122 of the computer 120 in FIG. 1A. The candidate tools may vary for each different type of candidate type of the interventional procedure for the anatomy. As a result, the anatomical characteristics and tool characteristics may individually or both vary for each different candidate type of the interventional procedure. As an example, a minimum width or height of an anatomical opening or passage may be compared with a diameter of a tool that would pass through or in the passage in a particular candidate type of the interventional procedure. Accordingly, S220 may involve comparing anatomical characteristics with tool characteristics of candidate tools to use in each of the multiple candidate types. By way of illustration and not limitation, examples of tool characteristics to be considered for suitability in a comparison with anatomical characteristics include: rigidity/flexibility of a tool, dexterity of tool components; or shape of the tool along its entire length or at the tip.

For therapeutic interventions, in particular ablation procedures, the size of the tool may be important and may be compared with anatomical characteristics at S220. For example, in ablation procedures, the diameter or length of an ablation needle may be compared with anatomical characteristics to determine whether an ablation needle is appropriate or whether ablation should be performed by a tool other than a needle.

At S230, anatomical movement is modelled. The modelling of anatomical movement at S230 may be performed by the controller 122 of the computer 120 in FIG. 1A. Alternatively, the modelling of anatomical movement at S230 may be performed outside of the system 100 in FIG. 1A and provided to the controller 122 of the computer 120 in FIG. 1A, such as based on pre-interventional imaging of the anatomy as it moves due to breathing or heartbeats. The modelling of anatomical movement at S230 may be based on actual observed movement such as breathing or heartbeats or based on expected movement that may have a varied impact for each of the different candidate types of the interventional procedure. The modelling at S230 may be incorporated into a feasibility report for each of the multiple candidate types as described below with respect to S240. Accordingly, S230 may involve modelling anatomical movement expected from each of the multiple candidate types of the interventional procedure. The modelling at S230 may include, for example, measurements of maximum expansion of parts of the anatomy during breathing or based on heartbeats, and the observation may be taken across multiple cycles such as 50 or 100 breaths.

For ablation, movement of the predicted ablation zone may be modelled at S230 and incorporated into the feasibility report at S240. For ablation, the modelling may be used to ensure that the ablation zone is properly located so that the proper tissue is removed in the ablation. The ablation zone may be predicted or identified as a function of both the tool characteristic as well as the motion of the tissue, and the prediction or identification may be made based on the relationship between tool and the motion of the tissue.

At S240, feasibility reports are generated for each of the multiple candidate types. The feasibility reports may be generated by the controller 122 of the computer 120 in FIG. 1A. The feasibility reports range from an aggregate score reflecting the feasibility of each candidate type to a range of qualitative and/or quantitative grades and/or scores for individual aspects of each candidate type. An aggregate score may be a feasibility grade weighted for each feasibility report. A feasibility grade is a numerical, alphabetical, or alphanumerical grade that characterizes a candidate type of interventional procedure based on the comparison(s) at S220 and that can be used to rank or otherwise rate a candidate type of interventional procedure against other candidate types of interventional procedure. Each feasibility report may include a feasibility grade. The feasibility grade may be a feasibility score, such as when the feasibility grade is solely numerical in nature. The weightings for the feasibility grade vary based on an expected diagnostic yield that varies for each of the different candidate types. Each feasibility report generated at S240 includes a feasibility grade that may be weighted differently for each of the multiple candidate types. For example, one candidate type of an interventional procedure may present unique risks relative to another candidate type and may be weighted at a lower weighting relative to the other candidate type as a result of the unique risks. Accordingly, S240 may involve generating, based on the identifying at S210 and the comparing at S220, a feasibility report for each of the multiple candidate types. Examples of the qualitative and/or quantitative characteristics that may be included in or reflected in a feasibility grade may include one or more of experience of operators who will perform the interventional procedure for each of the multiple candidate types; relative location of a target location for the interventional procedure in the anatomy; and patient health characteristics of a patient subject to the interventional procedure. The feasibility grade weighted for each feasibility report may vary from other feasibility reports based on the qualitative and/or quantitative characteristics included in or reflected in the feasibility reports.

Feasibility reports generated at S240 may also define interventional tool(s) that are predicted to successfully reach a target site in the intervention for the optimal interventional procedure type. For example, endobronchial biopsy may be the candidate type selected and a 5F catheter may be identified as the best interventional tool to use to reach the biopsy site. Tool characteristics may be used as inputs to both select the candidate type of interventional procedure, as well as the optimal interventional tool(s) to use in the interventional procedure. If more than one tool is selected, the order in which the tools will be used may also be included in the feasibility report.

In an embodiment, multiple feasibility reports may be generated for a single candidate type. For example, when multiple potential paths to a target location exist for a candidate type, each potential path may be provided with its own feasibility report. In another example, when multiple different tools may be used in alternative scenarios to reach a target of the interventional procedure, each different tool or viable combination of tools may be provided with its own feasibility report. The feasibility report with the highest feasibility grade(s) and/or scores may be selected as the feasibility report for the single candidate type to be compared with feasibility reports for other candidate types for a selection at S260 as described below.

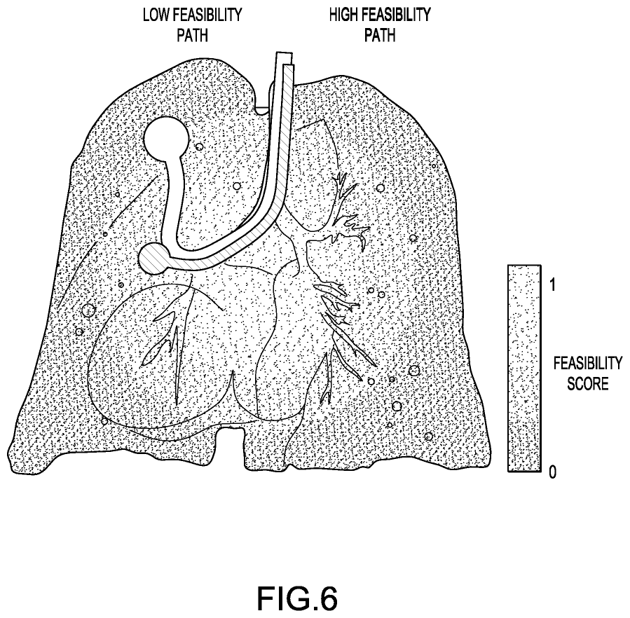

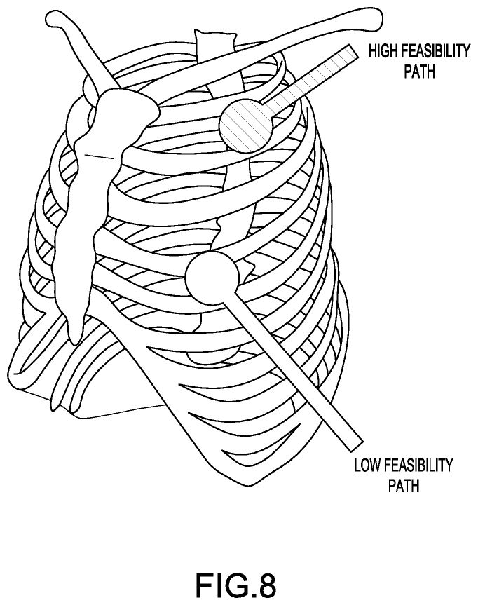

At S250, a heat map, which may be used for variety of purposes, is generated. The heat map may be generated by the controller 122 of the computer 120 in FIG. 1A and displayed on the display 130 in FIG. 1A. For example, a heat map may show feasibility of multiple paths to at least one target of the interventional procedure in the anatomy. Another heat map may show relative risks differentiating intervention with different tissues in the anatomy. The heat maps may visually reflect the feasibility reports or aspects of the feasibility reports generated at S240. As an example, a feasibility report may be based on or consist of a feasibility heat map across the entire lung. For example, the feasibility heat maps of FIGS. 6 and 8 described below may be color-coded to demonstrate higher risk biopsy plans with the color red, and lower risk biopsy plans with the color blue. In another embodiment, data from prior biopsy cases from similar patients with similar anatomy may be used to prepare such heat maps at S250.

At S260, an optimal interventional procedure type is selected based on the highest qualitative and/or quantitative grades and/or scores in the feasibility reports generated at S240. The selection at S260 may be made by the controller 122 of the computer 120 in FIG. 1A. Accordingly, S260 may involve selecting, based on the feasibility report for each of the multiple candidate types, an optimal intervention procedure type among the multiple candidate types. The selection at S260 may also include a selection of a candidate tool for the optimal interventional procedure. For example, a tool may be appropriate for one type of interventional procedure and not another. The selection at S260 may incorporate consideration of different tools to result in a selection of a candidate tool suitable for the optimal interventional procedure.

At S270, an interventional procedure is performed on the anatomy using the optimal interventional procedure type selected at S260. The interventional procedure may be a biopsy or a therapeutic type of interventional procedure, such as ablation. The interventional procedure may be performed under the guidance of the system 100 in FIG. 1A. The interventional procedure performed at S270 may be performed immediately after the steps from S210 to S260 or may be performed later such as on a later day, a later week, or even a later month. An example of the interventional procedure is a particular type of lung biopsy selected as the optimal interventional procedure type at S260. During the interventional procedure, interventional imagery such as x-ray or ultrasound imagery may be displayed on the display 130, for various purposes such as to confirm that the tool used in the interventional procedure has the expected or predicted shape. Alternatively, or additionally the imagery displayed on display 130 may be used to confirm mechanical characteristics with respect to the anatomy.

At S280, a clinical outcome of the interventional procedure performed at S270 is fed back to an artificial intelligence engine. The clinical outcome and other information relating to the interventional procedure may be fed back to the AI system 140 by the controller 122 of the computer 120 in FIG. 1A. The clinical outcome may be determined immediately or well after the interventional procedure. For example, confirmation of the success of an interventional procedure as a clinical outcome may take weeks or even months, so that the clinical outcome indicating success may require a delay before being fed back to the AI engine 142 of the AI system 140 in FIG. 1A. Characteristics used to generate subsequent feasibility reports may be based on output from the AI engine 142 based on previous clinical outcomes of interventional procedures. Accordingly, S280 may involve feeding back a clinical outcome from the interventional procedure to an AI system 140, and the feedback is incorporated into artificial intelligence to make future selections as in S260. For example, the computer 120 may store and execute an AI application that interfaces with and interacts with the AI system 140. An AI application on the computer 120 may be used to provide inputs to the AI system 140 so that the AI system 140 makes the selections at S260 or in order to obtain updated algorithms and/or updated models from the AI system 140 based on the artificial intelligence implemented by the AI system 140 so that the controller 122 of the computer 120 makes the selections at S260. The AI system 140 may be used interactively to select an optimal interventional procedure type and optimal tool based on previous instantiations of interventional procedures. For example, in an embodiment, information available from prior biopsies from patients, corresponding success/failure results, and actual risk measurements can be used to train a model for prediction of feasibility scores. A feasibility score may consist of a single number for each nodule or other target within the patient, and a separate feasibility score may be provided for each path through the anatomy being considered including each of multiple paths to the same target. For example, the higher the number of the feasibility score, the greater the likelihood of a successful clinical outcome. A logistical regression may be used to map success/failure results and actual risk measurements to determine proper weighting of inputs in generating feasibility scores. The training of an AI engine 142 in the AI system 140 may also be performed using data from patients with similar criteria such as nodule size, location, anatomical size, family history, to ensure the score calculation is as realistic as possible.

The feedback at S280 may also include feedback from the medical intervention, such as from sensors (not shown) on sensor-equipped tools. Sensors can be used to determine the three-dimensional location of part or all of the tool during an interventional procedure; to provide real-time images of the anatomy during the interventional procedure; to quantify tissue characteristics during the interventional procedure; or to provide mechanical feedback during the interventional procedure; or a combination thereof. The data collected during the interventional procedure from sensors on a sensor-equipped tool may be fed back to an AI system 140 for analysis of whether the selected interventional procedure type and tools produced the desired outcome. Deviations from expectations, such as deviations from a predicted path or expected tool shape can be used as feedback to the AI engine 142 to improve the tool predictions and feasibility reports. Force or pressure sensors may provide an indication of whether the tool is interacting with the tissue in an undesirable way.

Interventional imagery may also be fed back at S280. For example, interventional imagery may confirm that a tool used in an interventional procedure produced the desired outcome. Interventional imagery may be fed back from an ultrasound system (not shown) or x-ray system (not shown) to the AI system 140 in FIG. 1A.

At S290, a model is trained based at least in part on the feedback from S280, as well as feedback from other interventional procedures. The model may be trained by the AI system 140 of FIG. 1A, and the training may be applied as artificial intelligence to select the optimal interventional procedure type at S260 for future instantiations of the interventional procedure optimization described herein. The model trained at S290 may also be trained based on the feasibility report for the optimal interventional procedure type selected at S260. In turn, the feasibility report for each of the different candidate types may be based on the same model that is trained at S290. Accordingly, S290 may involve training a model based on the feasibility report for the optimal interventional procedure type and a clinical outcome from the interventional procedure. The model that is trained at S290 may be trained based on feasibility reports and clinical outcomes for multiple patients and constrained by similarities in or of at least one health characteristic of the multiple patients.

An interface or connection between the AI system 140 and the controller 122 may allow the controller 122 to obtain the model so as to perform operations in FIG. 2 such as S220, S240 and S260. Alternatively, several operations such as S220, S240 and/or S260 may be performed by the AI system 140 or a software program executed on the computer 120 that communicates with the AI system 140.

Additionally, many tools used in interventional procedures have smart sensing capabilities. Smart sensing is automated sensing by sensors on or in sensor-equipped tools. Sensor-equipped tools can be used to determine the three-dimensional location of part or all of the tool. For example, sensors on a tool can be used to track location of the tool via electromagnetic tracking or optical shape sensing. Sensor-equipped tools can also be used to provide real-time images of the anatomy. For example, sensors on a tool may be used for imaging including ultrasound, optical coherence tomography and x-ray. Sensor-equipped tools also may be used to quantify tissue characteristics. For example, sensors on a tool may be used to quantify tissue characteristics in diffuse reflectance spectroscopy or Raman spectroscopy. Sensor-equipped tools also may be used to provide mechanical feedback. For example, sensors on a tool may include force sensors or pressure sensors. Any of these sensors or combinations of sensors may also be used as feedback to the AI engine 142. For example, a tool equipped with optical shape sensing technology can continuously provide information about its 3D shape and location. This information can be compared to the predicted shape and path that tool should have taken, and any deviations from the predicted path or shape can be used as feedback to the AI engine 142 to improve the tool predictions and feasibility reports. Force or pressure sensors also may provide an indication of whether the tool is interacting with the tissue in an undesirable way. Readings from sensors on sensor-equipped tools may be provided as feedback to the AI engine 142 to improve the prediction of candidate tool performance and hence the feasibility report. That is, characteristics of sensor-equipped tools and data sensed by the sensors of the sensor-equipped tools may be included in input to the AI engine 142.

After the model is trained at S290, the method of FIG. 2 returns to S210 to identify anatomical characteristics for another interventional procedure optimization.

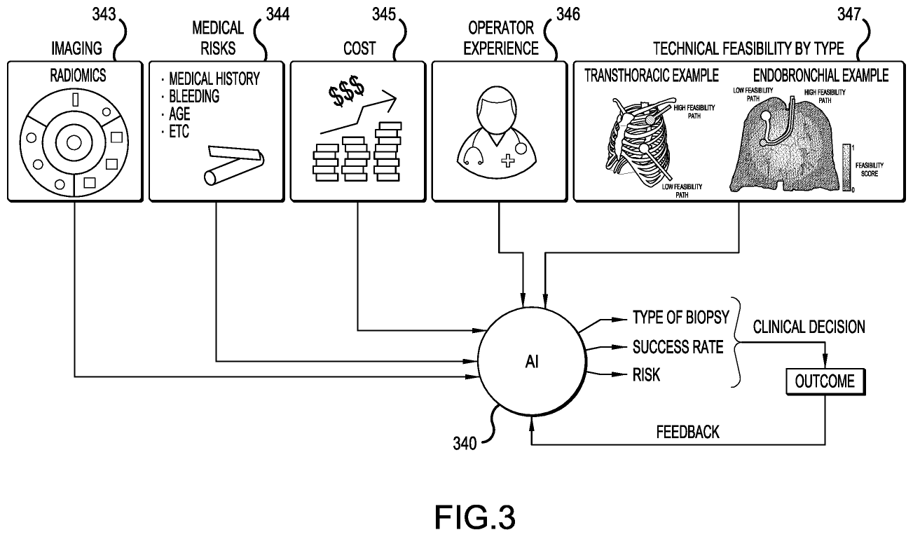

FIG. 3 illustrates an artificial intelligence overview for interventional procedure optimization, in accordance with a representative embodiment.

As shown in FIG. 3, an artificial intelligence system 340 receives as inputs multiple different types of data from multiple modalities, outputs a clinical decision to be used as a determination for a type of interventional procedure to perform and receives feedback of an outcome of the interventional procedure once performed. The artificial intelligence system 340 may be the AI system 140 in FIG. 1A, though the artificial intelligence system may be remote from or proximate to the computer 120 in FIG. 1A. In an embodiment, the artificial intelligence system 340 is fully or partially implemented in the computer 120 in FIG. 1A, so that a medical professional can obtain a recommendation from the artificial intelligence system 340 directly from the computer 120 in FIG. 1A. In other embodiments, the computer 120 may provide one or more of the multiple different types of data from the multiple modalities to the artificial intelligence system 340 and obtain a clinical decision from the artificial intelligence system 340. In yet other embodiments, the artificial intelligence described herein is distributed between the computer 120 and the artificial intelligence system 340. The artificial intelligence system 340 may include an artificial intelligence engine that is software based, as discussed above with regard to AI engine 142, for example. For example, the AI engine 142 may be implemented in the artificial intelligence system 340. The clinical decision output from the artificial intelligence system 340 may stratify patients into groups corresponding to the types of interventional procedures optimally selected based on the multi-modal input.

The artificial intelligence system 340 integrates the multi-modal data to generate the clinical decision as output. The multi-modal data may be provided to the artificial intelligence system 340 by the computer 120 in FIG. 1A as well as from other sources (not shown). The multi-modal data shown in FIG. 3 includes, but is not limited to, imaging 343, medical risks 344, procedure cost 345, operator experience 346, and technical feasibility 347 by type. The imaging 343 may be pre-interventional imagery, such as computed tomography imaging, MR imaging or ultrasound imaging. The imaging 343 may also be or include interventional imaging such as x-ray or ultrasound imaging. Interventional imaging as the imaging 343 may be used for confirmation that a tool used had the correctly predicted shape or mechanical characteristics with respect to the anatomy. The medical risks 344 are risks associated with each type of interventional procedure, such as the risk of damaging an important part of anatomy. The procedure cost 345 includes monetary costs for performing the procedure, such as services of skilled professionals, use of particular equipment and/or a facility, and costs of recovery such as use of a facility for recovery. The operator experience 346 is the experience of the personnel performing the interventional procedure and may be based on time spent performing similar procedures or a number of similar procedures performed by the personnel. Technical feasibility 347 is an assessment of each type of interventional procedure, such as types of biopsy, with respect to a patient's specific anatomy and condition. A risk score can be produced from the technical feasibility 347 by the artificial intelligence system 340. For example, if a nodule or other target is very close to a blood vessel, the chances of hitting the vessel during the biopsy, either because the planned path runs through the vessel or because the vessel sits directly behind the nodule or other target, may be very high. The output of the artificial intelligence system 340 may be a quantification of the feasibility for each type of interventional procedure being considered, including the corresponding success rate and risk factor score. The success rate may be determined using a historical database of similar interventional procedure types. The risk factor score may be based on success rates, failure rates, and complication rates for different types of complications arising from performance of similar interventional procedure types. The clinical decision may be made for each patient based on the quantification. Feeding back the resulting clinical outcome may help ensure continual performance improvement.

Applying artificial intelligence from the artificial intelligence system 340 may be used to determine feasibility of different potential types of interventional procedures for a specific circumstance, where the determined feasibility is provided in a feasibility report. In the example of biopsy, a feasibility score for each nodule that requires a biopsy may be generated for each biopsy method. The feasibility report may include the feasibility score calculated for each biopsy procedure based on the pre-interventional imagery of the imaging 343 and the data from the pre-interventional imagery as well as the information from one or more of the other modes of input to the artificial intelligence system 340. The data from the imagery may include, but is not limited to, the nodule location, anatomical structures containing or in the vicinity of the nodule, blood vessels, bony structures, organs at risk. Other data that may contribute to the feasibility score includes lab results such as from a blood test as well as procedure-specific data such as a gauge of a biopsy needle.

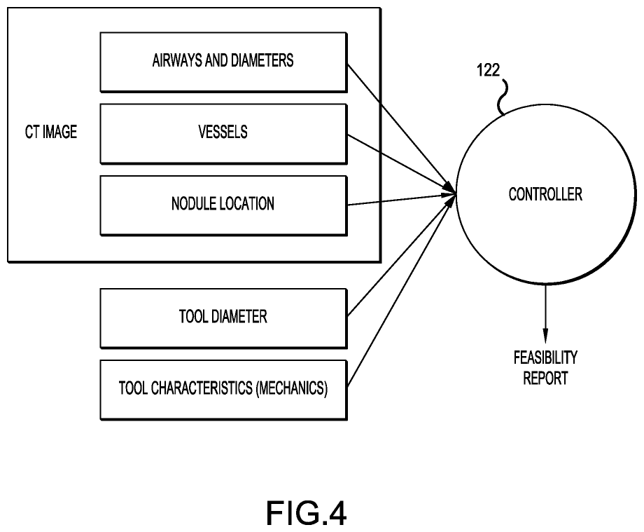

FIG. 4 illustrates a controller for generating a feasibility report for interventional procedure optimization, in accordance with a representative embodiment.

In FIG. 4, the controller 122 receives pre-interventional imagery of anatomy along with tool characteristics of candidate tools that may be used for each of the candidate types of interventional procedure. The controller 122 identifies anatomical characteristics from the pre-interventional imagery of anatomy for each of the multiple candidate types of the interventional procedure for the anatomy. The controller 122 also compares the anatomical characteristics with the tool characteristics of the candidate tools to use in each of the multiple candidate types. The comparison may involve dimensions of anatomy and dimensions of tools, for example.

In an embodiment, the interventional procedure is a lung biopsy and the pre-interventional imagery is computed tomography (CT) imagery. For an endobronchial biopsy of lung tissue, the computed tomography imagery may be used to find the airways and calculate diameter of the airways at different points, find the vessels relative to the airways and nodule, and find the nodule location. The airways and diameter of the airways, the vessels, and the nodule location may be extracted manually or by utilizing computer-aided tools that perform automated image analysis. The dimensions and mechanical properties of anatomy serve as input to a feasibility calculator implemented by the controller 122. A list of candidate biopsy tools is also provided to the controller 122, including details of the biopsy tools including tool dimensions such as lengths, widths and diameters and tool mechanical properties. For example, the maximum amount of curvature or bending ability of a tool, the steerability of a tool, and other similar types of details may be used to determine whether the tool can mechanically be moved through the airways. As another example, a feasibility calculator implemented by the controller 122 may calculate the shortest path from the trachea to the nodule via the airways.

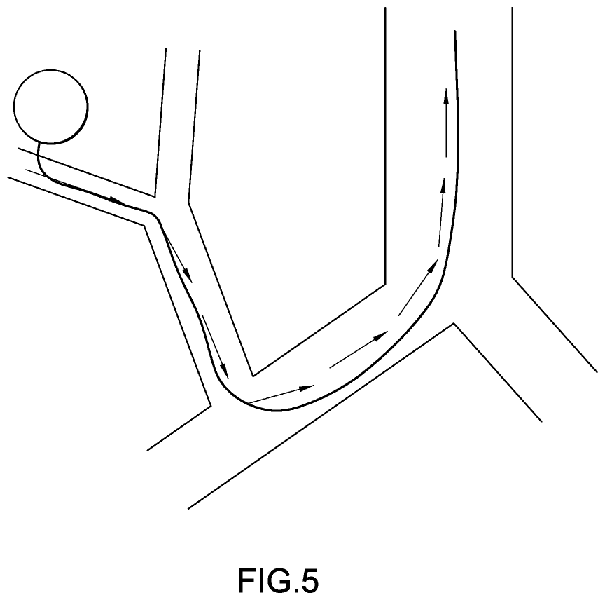

FIG. 5 illustrates path planning for an interventional procedure in interventional procedure optimization, in accordance with a representative embodiment.

In FIG. 5, a shortest path to a target of an interventional procedure is calculated, such as by the controller 122 in FIG. 4 or the controller 122 in the computer 120 in FIG. 1A. Path planning may be performed by starting at a nodule, locating the airway closest to the nodule, and proceeding through the airways until the trachea is reached. The diameter of the airways and the maximum amount of curvature in the planned path is then compared to the available tool dimensions, such as diameter and bendability, of the various tools needed for the type of interventional procedure. From this a feasibility score may be generated for the nodule, as in S240 in FIG. 2. The path planning in FIG. 5 may be performed for multiple different paths in a single type of interventional procedure, and/or for multiple different types of interventional procedures. For example, one type of interventional procedure may involve passage through a first part of the anatomy but not a second part of the anatomy and another type of interventional procedure may involve passage through the second part of the anatomy but not the first part of the anatomy.

FIG. 6 illustrates a feasibility map for interventional procedure optimization, in accordance with a representative embodiment.

FIG. 6 shows an example of two feasibility scores for the endobronchial biopsy in the example used for FIG. 6. If the nodule is in a peripheral part of the lung which is difficult to reach with the available tools and small airways, then the feasibility score is considered low. However, if the nodule is close to the trachea and if multiple different biopsy tools can be used, then the feasibility score is considered high.

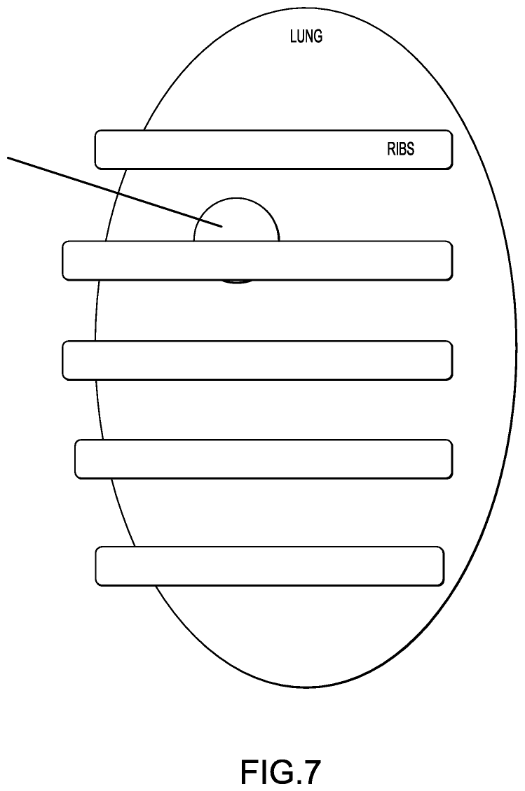

The controller 122 of FIGS. 1A and 4 may also be used to generate a feasibility report for other types of interventional procedures. For example, transthoracic biopsy of lung tissue may be proposed for a lung biopsy. Pre-operative imagery such as computed tomography imagery may be analyzed to find the location of the nodule, vessels, and rib cage and organs on the path to the nodule which serve as inputs to the feasibility calculator implemented by the controller 122. The available tool sizes and mechanics may also be used as inputs to the controller 122. For example, it may be important to know the distance a biopsy needle proceeds into the tissue after it is “fired,” such as to avoid overshooting when an overshot needle may puncture a critical tissue and/or organ. The travel distance of a biopsy needle may be important for nodules close to the heart or major blood vessels. The information from the computed tomography imagery may be used to calculate an optimal planned path to the nodule. Again, a feasibility score may be provided for each nodule, as explained below with respect to FIG. 7.

FIG. 7 illustrates path planning for another interventional procedure in interventional procedure optimization, in accordance with a representative embodiment.

As shown in FIG. 7, a model of a lung is used to project a path between ribs for a transthoracic needle to reach a target location (nodule). The model of the lung may be a generic model applicable to multiple or even most patients or may be customized with anatomical details of a particular patient. The path planning of FIG. 7 may be implemented by the controller 122 of the computer 120 in FIG. 1A.

FIG. 8 illustrates another feasibility map for interventional procedure optimization, in accordance with a representative embodiment.

FIG. 8 shows two nodules with different feasibility scores based on the path planning from FIG. 7. A nodule that is close to the periphery of the lung has a high feasibility score because it is close to the rib cage. A nodule that is deep within the lung and sitting behind a rib may be more difficult to reach with the available biopsy needles and therefore has a low feasibility score.

An analysis similar to those provided above for the endobronchial biopsy (FIGS. 4, 5 and 6) and the transthoracic biopsy (FIGS. 7 and 8) may be provided for a surgical biopsy. The analysis may be performed by the controller 122 of the computer 120 in FIG. 1A. In all three biopsy approaches, respiratory motion or lung deflation may also be modeled as in S230 in FIG. 2. This motion modelling may serve as an additional input to the feasibility calculators implemented by the controller 122 for better prediction of technical feasibility.

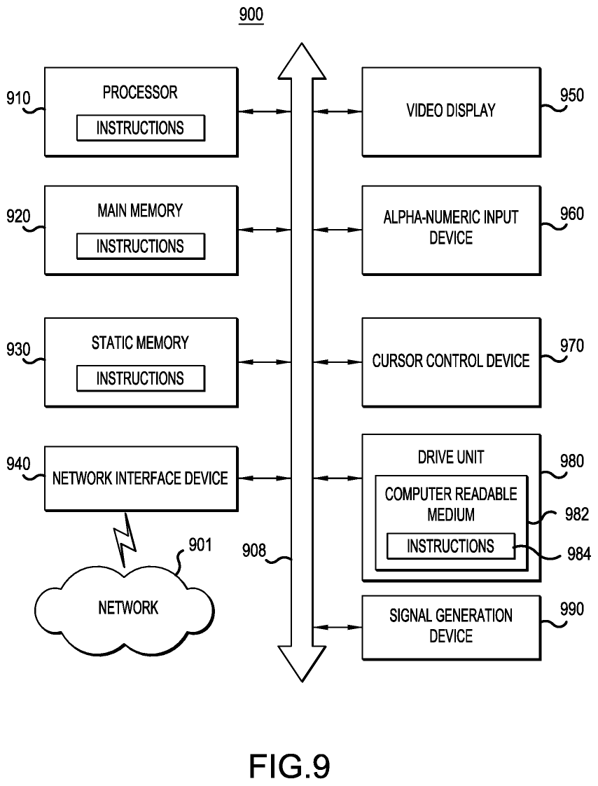

FIG. 9 illustrates a computer system, on which a method for interventional procedure optimization is implemented, in accordance with another representative embodiment.

The computer system 900 of FIG. 9 shows a complete set of components for a communications device or a computer device. However, a “controller” as described herein may be implemented with less than the set of components of FIG. 9, such as by a memory and processor combination. The computer system 900 may include some or all elements of one or more component apparatuses in a system for interventional procedure optimization herein, although any such apparatus may not necessarily include one or more of the elements described for the computer system 900 and may include other elements not described.

Referring to FIG. 9, the computer system 900 includes a set of software instructions that can be executed to cause the computer system 900 to perform any of the methods or computer-based functions disclosed herein. The computer system 900 may operate as a standalone device or may be connected, for example, using a network 901, to other computer systems or peripheral devices. In embodiments, a computer system 900 performs logical processing based on digital signals received via an analog-to-digital converter.

In a networked deployment, the computer system 900 operates in the capacity of a server or as a client user computer in a server-client user network environment, or as a peer computer system in a peer-to-peer (or distributed) network environment. The computer system 900 can also be implemented as or incorporated into various devices, such as the computer 120 in FIG. 1A, a stationary computer, a mobile computer, a personal computer (PC), a laptop computer, a tablet computer, or any other machine capable of executing a set of software instructions (sequential or otherwise) that specify actions to be taken by that machine. The computer system 900 can be incorporated as or in a device that in turn is in an integrated system that includes additional devices. In an embodiment, the computer system 900 can be implemented using electronic devices that provide voice, video or data communication. Further, while the computer system 900 is illustrated in the singular, the term “system” shall also be taken to include any collection of systems or sub-systems that individually or jointly execute a set, or multiple sets, of software instructions to perform one or more computer functions.

As illustrated in FIG. 9, the computer system 900 includes a processor 910. The processor 910 may be considered a representative example of the processor 12210 of the controller 122 in FIG. 1B and executes instructions to implement some or all aspects of methods and processes described herein. The processor 910 is tangible and non-transitory. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The processor 910 is an article of manufacture and/or a machine component. The processor 910 is configured to execute software instructions to perform functions as described in the various embodiments herein. The processor 910 may be a general-purpose processor or may be part of an application specific integrated circuit (ASIC). The processor 910 may also be a microprocessor, a microcomputer, a processor chip, a controller, a microcontroller, a digital signal processor (DSP), a state machine, or a programmable logic device. The processor 910 may also be a logical circuit, including a programmable gate array (PGA), such as a field programmable gate array (FPGA), or another type of circuit that includes discrete gate and/or transistor logic. The processor 910 may be a central processing unit (CPU), a graphics processing unit (GPU), or both. Additionally, any processor described herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems. The term computing device should also be interpreted to include a collection or network of computing devices each including a processor or processors. Programs have software instructions performed by one or multiple processors that may be within the same computing device or which may be distributed across multiple computing devices.

The computer system 900 further includes a main memory 920 and a static memory 930, where memories in the computer system 900 communicate with each other and the processor 910 via a bus 908. Either or both of the main memory 920 and the static memory 930 may be considered representative examples of the memory 12220 of the controller 122 in FIG. 1B, and store instructions used to implement some or all aspects of methods and processes described herein. Memories described herein are tangible storage mediums for storing data and executable software instructions and are non-transitory during the time software instructions are stored therein. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The main memory 920 and the static memory 930 are articles of manufacture and/or machine components. The main memory 920 and the static memory 930 are computer-readable mediums from which data and executable software instructions can be read by a computer (e.g., the processor 910). Each of the main memory 920 and the static memory 930 may be implemented as one or more of random access memory (RAM), read only memory (ROM), flash memory, electrically programmable read only memory (EPROM), electrically erasable programmable read-only memory (EEPROM), registers, a hard disk, a removable disk, tape, compact disk read only memory (CD-ROM), digital versatile disk (DVD), floppy disk, blu-ray disk, or any other form of storage medium known in the art. The memories may be volatile or non-volatile, secure and/or encrypted, unsecure and/or unencrypted.

“Memory” is an example of a computer-readable storage medium. Computer memory is any memory which is directly accessible to a processor. Examples of computer memory include, but are not limited to RAM memory, registers, and register files. References to “computer memory” or “memory” should be interpreted as possibly being multiple memories. The memory may for instance be multiple memories within the same computer system. The memory may also be multiple memories distributed amongst multiple computer systems or computing devices.

As shown, the computer system 900 further includes a video display unit 950, such as a liquid crystal display (LCD), an organic light emitting diode (OLED), a flat panel display, a solid-state display, or a cathode ray tube (CRT), for example. Additionally, the computer system 900 includes an input device 960, such as a keyboard/virtual keyboard or touch-sensitive input screen or speech input with speech recognition, and a cursor control device 970, such as a mouse or touch-sensitive input screen or pad. The computer system 900 also optionally includes a disk drive unit 980, a signal generation device 990, such as a speaker or remote control, and/or a network interface device 940.

In an embodiment, as depicted in FIG. 9, the disk drive unit 980 includes a computer-readable medium 982 in which one or more sets of software instructions 984 (software) are embedded. The sets of software instructions 984 are read from the computer-readable medium 982 to be executed by the processor 910. Further, the software instructions 984, when executed by the processor 910, perform one or more steps of the methods and processes as described herein. In an embodiment, the software instructions 984 reside all or in part within the main memory 920, the static memory 930 and/or the processor 910 during execution by the computer system 900. Further, the computer-readable medium 982 may include software instructions 984 or receive and execute software instructions 984 responsive to a propagated signal, so that a device connected to a network 901 communicates voice, video or data over the network 901. The software instructions 984 may be transmitted or received over the network 901 via the network interface device 940.

In an embodiment, dedicated hardware implementations, such as application-specific integrated circuits (ASICs), field programmable gate arrays (FPGAs), programmable logic arrays and other hardware components, are constructed to implement one or more of the methods described herein. One or more embodiments described herein may implement functions using two or more specific interconnected hardware modules or devices with related control and data signals that can be communicated between and through the modules. Accordingly, the present disclosure encompasses software, firmware, and hardware implementations. Nothing in the present application should be interpreted as being implemented or implementable solely with software and not hardware such as a tangible non-transitory processor and/or memory.

In accordance with various embodiments of the present disclosure, the methods described herein may be implemented using a hardware computer system that executes software programs. Further, in an exemplary, non-limited embodiment, implementations can include distributed processing, component/object distributed processing, and parallel processing. Virtual computer system processing may implement one or more of the methods or functionalities as described herein, and a processor described herein may be used to support a virtual processing environment.

Accordingly, interventional procedure optimization enables automated determinations for an optimized type of an interventional procedure such as a biopsy of a lung. Nevertheless, interventional procedure optimization is not limited as an application to lungs, and instead is applicable to other organs for which multiple biopsy approaches may be feasible. Similarly, interventional procedure optimization is not limited to biopsies, and instead is applicable to other types of interventional procedures such as ablation or other types of therapeutic interventions in which multiple approaches may be feasible.

Although interventional procedure optimization has been described with reference to several exemplary embodiments, it is understood that the words that have been used are words of description and illustration, rather than words of limitation. Changes may be made within the purview of the appended claims, as presently stated and as amended, without departing from the scope and spirit of interventional procedure optimization in its aspects. Although interventional procedure optimization has been described with reference to particular means, materials and embodiments, interventional procedure optimization is not intended to be limited to the particulars disclosed; rather interventional procedure optimization extends to all functionally equivalent structures, methods, and uses such as are within the scope of the appended claims.

The illustrations of the embodiments described herein are intended to provide a general understanding of the structure of the various embodiments. The illustrations are not intended to serve as a complete description of all of the elements and features of the disclosure described herein. Many other embodiments may be apparent to those of skill in the art upon reviewing the disclosure. Other embodiments may be utilized and derived from the disclosure, such that structural and logical substitutions and changes may be made without departing from the scope of the disclosure. Additionally, the illustrations are merely representational and may not be drawn to scale. Certain proportions within the illustrations may be exaggerated, while other proportions may be minimized. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

1. A controller for interventional procedure optimization, comprising:
a memory that stores instructions; and
a processor that executes the instructions, wherein, when executed by the processor, the instructions cause the controller to implement a process that includes:
identifying anatomical characteristics from pre-interventional imagery of anatomy for each of a plurality of candidate types of interventional procedures for the anatomy;
comparing the anatomical characteristics with tool characteristics of each of a plurality of candidate tools to use in each of the plurality of candidate types;
generating, based on the identifying and the comparing, a feasibility report for each of the plurality of candidate types of interventional procedure, each feasibility report including a feasibility grade indicative of a ranking of the candidate type of interventional procedure, and
selecting, based on the feasibility report for each of the plurality of candidate types, an optimal interventional procedure type among the plurality of candidate types, wherein an interventional procedure is performed on the anatomy using the optimal interventional procedure type based on the selecting
and wherein the feasibility report for the optimal interventional procedure type further defines which of the plurality of candidate tools is predicted to reach a target site in the optimal interventional procedure type.

2. The controller of claim 1,
wherein the process implemented when the processor executes the instructions further includes automatically detecting a target location for the interventional procedure as one of the anatomical characteristics identified from identifying anatomical characteristics from pre-interventional imagery of anatomy, wherein the plurality of candidate types of the interventional procedure include an endobronchial biopsy of lung tissue through airways, a transthoracic biopsy of lung tissue through a thoracic cavity, and a surgical biopsy of the lung tissue performed by surgery.

3. The controller of claim 1, wherein the process implemented when the processor executes the instructions further includes:
generating, based on the identifying and comparing, a plurality of feasibility reports for one candidate type of the plurality of candidate types of the interventional procedure using different paths to a target of the interventional procedure, and
selecting, based on the feasibility reports for each different path, the feasibility report for the one candidate type of the plurality of candidate types to be compared with the feasibility report for each other of the plurality of candidate types for a selection of the optimal interventional procedure type.

4. The controller of claim 1, wherein the process implemented when the processor executes the instructions further includes:
generating, based on the identifying and comparing, a plurality of feasibility reports for one candidate type of the plurality of candidate types of the interventional procedure using different tools to reach a target of the interventional procedure, and
selecting, based on the feasibility reports for each different tool, the feasibility report for the one candidate type of the plurality of candidate types to be compared with the feasibility report for each other of the plurality of candidate types for a selection of the optimal interventional procedure type.

5. The controller of claim 1, wherein the feasibility grade weighted for each feasibility report varies based on at least one of experience of operators who will perform the interventional procedure for each of the plurality of candidate types, relative location of a target location for the interventional procedure in the anatomy, or patient health characteristics of a patient subject to the interventional procedure.

6. The controller of claim 1, wherein weightings for the feasibility grade vary for each of the plurality of candidate types of the interventional procedure based on an expected diagnostic yield that varies for each of the plurality of candidate types.

7. The controller of claim 1, wherein the process implemented when the processor executes the instructions further includes feeding back a clinical outcome from the interventional procedure to an artificial intelligence engine, wherein characteristics used to generate each feasibility report are based on output from the artificial intelligence engine based on previous clinical outcomes of interventional procedures, and wherein characteristics of sensor-equipped tools are included in input to the artificial intelligence engine from the previous clinical outcomes.

8. The controller of claim 1,
wherein the anatomical characteristics include at least one of a diameter of an airway, a curvature of the airway, an elasticity of an airway or an elasticity of tissues surrounding the anatomy subject to the interventional procedure.

9. The controller of claim 1,
wherein the anatomical characteristics include a relative location of a target of the interventional procedure in the anatomy and a path to the relative location of the target of the interventional procedure.

10. The controller of claim 1,
wherein the process implemented when the processor executes the instructions further includes generating a heat map showing feasibility of a plurality of paths to at least one target of the interventional procedure in the anatomy.

11. The controller of claim 1,
wherein the process implemented when the processor executes the instructions further includes generating a heat map showing relative risks differentiating intervention with different tissues in the anatomy.

12. The controller of claim 1, wherein the process implemented when the processor executes the instructions further includes:
modelling anatomical movement expected from each of the plurality of candidate types of the interventional procedure; and
incorporating the modelling into the feasibility report for each of the plurality of candidate types.

13. The controller of claim 1, wherein the process implemented when the processor executes the instructions further includes training a model based on the feasibility report for the optimal interventional procedure type and a clinical outcome from the interventional procedure, wherein the feasibility report for each of the plurality of candidate types is based on the model.

14. The controller of claim 13, wherein the model is trained based on feasibility reports and clinical outcomes for a plurality of patients and constrained by similarity in at least one health characteristic for the plurality of patients.

15. The controller of claim 1, wherein the interventional procedure comprises a biopsy, and the plurality of candidate types of the interventional procedure comprises biopsy types.

16. (canceled)

17. (canceled)

18. (canceled)

19. (canceled)

20. (canceled)
